(19) United States
(12) Patent Application Publication
(10) Pub. No.: US 2022/0327696 A1
(43) Pub. Date: Oct. 13, 2022

(54) LIGHT IRRADIATION DEVICE AND LIGHT IRRADIATION METHOD

(71) Applicants: Takuya Nagano, Kanagawa (JP); Tsubasa Tamura, Kanagawa (JP); Kasumi Nakamura, Kanagawa (JP); Shun Okazaki, Kanagawa (JP); Mitsuo Suzuki, Kanagawa (JP)

(72) Inventors: Takuya Nagano, Kanagawa (JP); Tsubasa Tamura, Kanagawa (JP); Kasumi Nakamura, Kanagawa (JP); Shun Okazaki, Kanagawa (JP); Mitsuo Suzuki, Kanagawa (JP)

(73) Assignee: Ricoh Company, Ltd., Tokyo (JP)

(21) Appl. No.: 17/657,764

(22) Filed: Apr. 4, 2022

(30) Foreign Application Priority Data
Apr. 9, 2021 (JP) 2021-066446

(51) Int. Cl. G06T 7/00

(52) U.S. Cl. CPC G06T 7/0012; G06T 2207/10004; G06T 2207/10024; G06T 2207/10048

(57) ABSTRACT

A light irradiation device includes a first illumination optical system to emit first light that causes a chemical reaction on an object; a second illumination optical system to emit second light having a wavelength different from a wavelength of the first light; a light emitter to emit the first light from the first illumination optical system and the second light from the second illumination optical system to the object in space shared by the first light and the second light, to irradiate an irradiation area on the object; an image-capturing optical system to capture an image of the space including the object; and circuitry configured to: detect a state of the irradiation area based on the image captured by the image-capturing optical system; and control at least one of the first light and the second light based on the detected state.

### CROSS-REFERENCE TO RELATED APPLICATIONS

This patent application is based on and claims priority pursuant to 35 U.S.C. § 119(a) to Japanese Patent Application No. 2021-066446, filed on Apr. 9, 2021, in the Japan Patent Office, the entire disclosure of which is hereby incorporated by reference herein.

### BACKGROUND

### Technical Field

The present disclosure relates to a light irradiation device and a light irradiation method.

### Related Art

Among light contained in sunlight, medium ultraviolet rays having wavelengths shorter than visible light or light in the ultraviolet (UV) UV-B region having wavelengths of 280 to 315 nm have an action of activating skin cells, and are used in the fields such as medicine and cosmetics. Near infrared rays having a wavelength longer than that of visible light have been used for, for example, improvement of blood circulation and relief of pain. Recently, such near infrared rays are expected to be used in photoimmunotherapy that utilizes a photochemical reaction between near-infrared rays and a photosensitive substance.

### SUMMARY

An embodiment provides a light irradiation device including a first illumination optical system to emit first light that causes a chemical reaction on an object; a second illumination optical system to emit second light having a wavelength different from a wavelength of the first light; a light emitter to emit the first light from the first illumination optical system and the second light from the second illumination optical system to the object in space shared by the first light and the second light, to irradiate an irradiation area on the object; an image-capturing optical system to capture an image of the space including the object; and circuitry configured to: detect a state of the irradiation area based on the image captured by the image-capturing optical system; and control at least one of the first light and the second light based on the detected state.

Another embodiment provides light irradiation method including capturing an image of a full field including an irradiation area on an object irradiated with first light and second light coaxially emitted to the object, the first light and the second light having wavelengths different from each other; detecting a state of the irradiation area based on the captured image; determining an orientation of each optical modulator of an optical modulator array that emits the first light and the second light to the object according to the state of the irradiation area detected in the detecting; controlling each optical modulator of the optical modulator array to have the orientation determined in the determining; controlling a first light source to emit the first light in a first direction and a second light source to emit the second light in a second direction to the optical modulator array with each optical modulator controlled to have the orientation determined in the determining; and emitting the first light reflected in a third direction by each optical modulator and the second light reflected in the third direction by each optical modulator to irradiate the object, the third direction being different from each of the first direction and the second direction.

### BRIEF DESCRIPTION OF THE DRAWINGS

A more complete appreciation of the disclosure and many of the attendant advantages and features thereof can be readily obtained and understood from the following detailed description with reference to the accompanying drawings, wherein:

FIG. 1 is an illustration of a configuration of a light irradiation device according to an embodiment;

FIG. 2 is a block diagram of a functional configuration of a control unit implemented by an electronic circuit in FIG. 1;

FIG. 3 is an illustration of a configuration of an optical system of a light irradiation device according to a first embodiment;

FIG. 4 is an illustration of an area irradiated with first light and another area irradiated with second light;

FIG. 5 is an illustration of a relative position between a first illumination optical system, a second illumination optical system, and a projection optical system;

FIG. 6 is an illustration of a healing process by using the light irradiation device according to an embodiment;

FIG. 7 is an illustration of a healing process by using the light irradiation device, according to another embodiment;

FIG. 8A is an illustration of a configuration that enables maximum amounts of the first light and the second light, according to an embodiment of the present disclosure;

FIG. 8B is an illustration of a configuration that enables an increase in the amount of the first light, according to an embodiment of the present disclosure;

FIG. 9A is an illustration of a configuration that is difficult to increase the amount of the first light, according to a comparative example;

FIG. 9B is an illustration of a configuration that is difficult to increase the amount of the first light, according to a comparative example;

FIG. 10 is an illustration of configurations of the optical systems of a light irradiation device according to a second embodiment; and

FIG. 11 is an illustration of configurations of the optical systems of a light irradiation device according to a third embodiment.

The accompanying drawings are intended to depict embodiments of the present invention and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described below. As used herein, the singular forms “a,” “an,” and “the” are intended to include the plural forms as well, unless the context clearly indicates otherwise.

A hand-held phototherapy device in which an ultraviolet light source is housed in a housing has been proposed. A typical phototherapy device using UV-B light (or medium ultraviolet light) only irradiates the skin with light emitted from a light source. In particular, in the case of irradiating a patchy pattern, regions other than the patchy region are irradiated with UV-B light, and the contrast of the skin increases due to ultraviolet burning, pigmentation, or the like, so that care must be taken in handling the device. When the intensity of the irradiation light is lowered, the irradiation efficiency is lowered.

For the use of near-infrared light in photoimmunotherapy, adverse effects such as skin damage and damage to muscle tissues have been recently attracting attentions. To deal with such adverse effects, the intended location is to be accurately irradiated. Since neither ultraviolet light nor infrared light can be seen by human eyes, visible light is to be used as a guide for a highly accurate irradiation, but this has not been implemented at present.

In order to prevent irradiation of an area other than the intended irradiation area, it is conceivable to manufacture a mask in which only the intended irradiation area is opened, but it takes time and effort to manufacture such a mask. Further, using such a mask involves recreating the mask each time after irradiation. Further, the shape of the patchy pattern is not uniform, and the shape of the mask and the irradiation amount are different for each irradiated object. Portions other than the intended region may be irradiated due to, for example, a deviation in the shape or size of the mask opening, or a positional deviation of the mask. Merely using such masks still fails to overcome the issues.

Embodiments of the present disclosure achieves photodynamic therapy on the intended area while reducing or preventing irradiation of an area other than the intended irradiation area.

Embodiments of the present disclosure enable an intended area to be irradiated with exposure dose sufficient to perform intended performance while reducing or preventing areas other than the intended area from being irradiated with light. Embodiments of the present disclosure further enable irradiation light to be guided to the affected area with a high accuracy, using second light having wavelengths different from those of the irradiation light. Hereinafter, the present disclosure will be described based on specific configuration examples. The same components are denoted by the same reference numerals, and redundant description may be omitted.

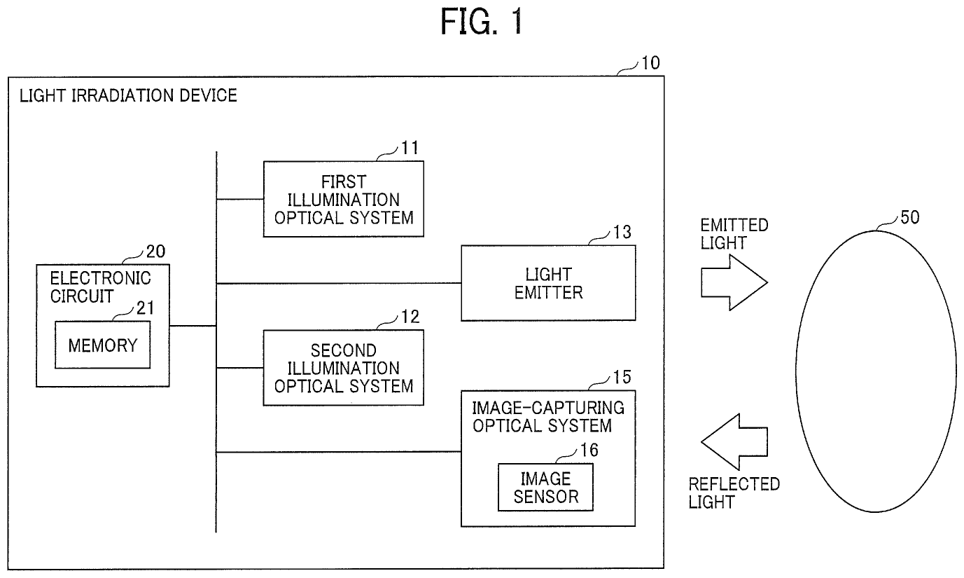

FIG. 1 is an illustration of a configuration of a light irradiation device 10 according to an embodiment. The light irradiation device 10 is used as a light treatment device. The light irradiation device 10 switches between ON and OFF irradiation states for each micro area of the irradiation area (the intended area to be irradiated) of an object 50 to be irradiated, to allow a high-accurate light irradiation on the intended area while reducing or preventing light irradiation on areas other than the intended area. Using such irradiation light and light guiding the irradiation light achieves a higher accuracy of the irradiation position.

The light irradiation device 10 includes a first illumination optical system 11, a second illumination optical system 12, a light emitter 13, an image-capturing optical system 15, and an electronic circuit 20. The first illumination optical system 11 emits first light to the object 50 to cause a photochemical reaction in the object 50 irradiated with the first light. The first light is used to irradiate the intended spot. Examples of the first light include ultraviolet light and infrared light. In this example, UV-B light is used as the first light.

The second illumination optical system 12 emits second light having a wavelength different from that of the first light. The second light is used as a guide guiding the first light to the intended spot. The second light is visible light (for example, red light).

The light emitter 13 projects (emits) the first light and the second light toward the object 50 to be irradiated, in the same space. The light emitter 13 switches between ON and OFF irradiation state for each micro area on the object 50 in response to a control signal from the electronic circuit 20. As will be described later, the light emitter 13 includes an optical modulator array in which multiple micro-optical modulators are arrayed. The optical modulators are respectively controlled to emit light beams so as to control irradiation for each micro area on the object 50.

The image-capturing optical system 15 includes an image sensor 16 such as a charge coupled device (CCD), a complementary metal oxide semiconductor (CMOS). Using such an image sensor 16, the image-capturing optical system 15 captures an image of space (a full field) including the object 50 to be irradiated. The image sensor 16 may have sensitivity to a visible light region or sensitivity to both visible light and UV-B light.

The electronic circuit 20 controls the operations of the first illumination optical system 11, the second illumination optical system 12, the light emitter 13, and the image-capturing optical system 15. The electronic circuit 20 may be a microprocessor with built-in memory, a logic device, or a field programmable gate array (FPGA). The electronic circuit 20 may include the memory 21 therein, or may be separate from the memory.

The electronic circuit 20 turns on the light sources of the first illumination optical system 11 and the second illumination optical system 12 in response to a command input to the light irradiation device 10, and controls the output level of irradiation light to be emitted from the light sources. The electronic circuit 20 also detects the state of the object 50 from the image acquired from the image-capturing optical system 15, and controls the operation of the light emitter 13 according to the detection result, or the detected state of the object 50.

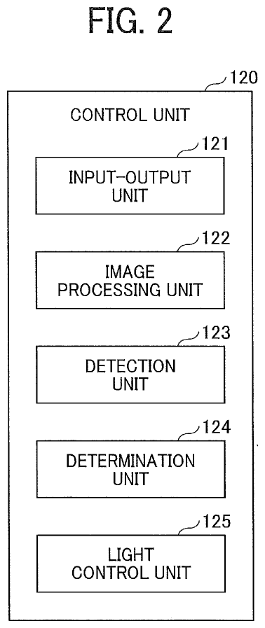

FIG. 2 is a block diagram of a functional configuration of a control unit 120 implemented by the electronic circuit 20. At least a part of the electronic circuit 20 serves as the control unit 120 of the light irradiation device 10. The control unit 120 includes an input-output unit 121, an image processing unit 122, a detection unit 123, a determination unit 124, and a light control unit 125. The input-output unit 121 inputs and outputs, for example, electrical signals and electronic data to and from an external device to allow communication between the light irradiation device 10 and the external device. The image processing unit 122 processes electrical signals output from the image-capturing optical system 15 to generate image data.

The detection unit 123 detects the state of the irradiated object 50, or information including the progress of irradiation and a skin state, from the image data generated by the image processing unit 122. The determination unit 124 determines the orientation (angle) or tilt of each optical modulator of the optical modulator array of the light emitter 13 based on information detected by the detection unit 123. The light control unit 125 controls the orientation or angle of each optical modulator so as to be the orientation or angle determined by the determination unit 124. Details of the orientation control of the optical modulators will be described later.

The light irradiation device 10 switches between ON and Off irradiation states for each micro region using the light emitter 13 to prevent an area other than the intended area from being irradiated with the emitted light while achieving a high-accuracy irradiation of the intended area. Further, using the first light serving as irradiation light as well as the second light serving as a guide enables acquisition of an image of the irradiated area and the visible light (i.e., the second light) as a guide and thus achieves a higher-accurate irradiation of the intended area while checking the irradiation position. Specific configurations of the optical systems will be described below.

### First Embodiment

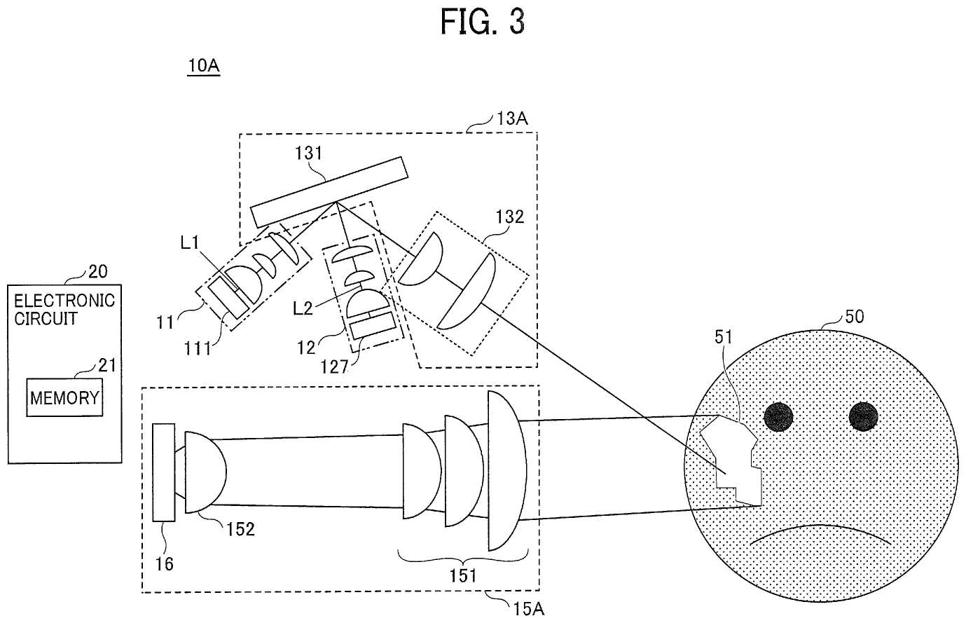

FIG. 3 is an illustration of the configurations of the optical systems of the light irradiation device 10A according to a first embodiment. In this example, the light irradiation device 10A is used as a light treatment device. The first illumination optical system 11 includes a light source 111 (a first light source), and emits first light L1 from the light source 111. The first light L1 is UV-B light in this example. The light source 111 is a solid-state light source such as a laser diode (LD) and a light emitting diode (LED). When the light source 111 is a light emitting diode, one or more lenses may be used to collimate the light into parallel light.

The second illumination optical system 12 includes a light source 127 (a second light source), and emits second light L2 from the light source 127. The second light L2 is visible light. The light source 127 is a solid-state light source such as a visible LED source and a visible light laser. When the light source 127 is a visible LED source, one or more lenses may be used to collimate the second light L2 into parallel light. The relative positions of the first illumination optical system 11 and the second illumination optical system 12 will be described later.

The light emitter 13A includes an optical modulator array 131 and a projection optical system 132. The optical modulator array 131 includes multiple micro optical modulators two-dimensionally arrayed. The optical modulators are micro-electromechanical systems (MEMS) such as digital micromirror devices (DMDs). Each of the optical modulators reflects the first light L1 and the second light L2 while switching its orientation, or angle at high speed.

Using the optical modulator array 131 with a two-dimensional array of multiple optical modulators enables adjustment of the irradiation level for each micro area of an affected area and achieves irradiation of the affected area with a higher accuracy unlike the case in which the affected area is uniformly irradiated. The light irradiation device 10A supports a patchy pattern such as a white patch, or the vitiligo patch.

The projection optical system 132 projects (emits) the first light L1 and the second light L2 toward an irradiation area 51 of the object 50 in a common space (or space shared by the first light and the second light). The term “project (emit) the first light and the light L2 . . . in a common space” refers to projecting (emitting) the first light L1 and the second light L2 along the same axis, or coaxially (an axis common between the first light L1 and the second light L2) to the irradiation area 51. The “same axis, or coaxially” refers to substantially the same axis and includes slight fluctuation of an optical path, slight deviation due to, for example, the influence of a refractive index.

For each optical modulator of the optical modulator array 131, the first light L1 and the second light L2 are not simultaneously reflected by each optical modulator, but alternately reflected by each optical modulator according to its angle, to the projection optical system 132. The operation speed of the optical modulators is so high that the human eyes recognize the first light L1 and the second light L2 to be coaxially projected from the projection optical system 132 at substantially the same time. The user of the light irradiation device 10A or the irradiated object recognizes the current irradiation position irradiated with the first light L1 by visually observing the second light L2 that is visible light.

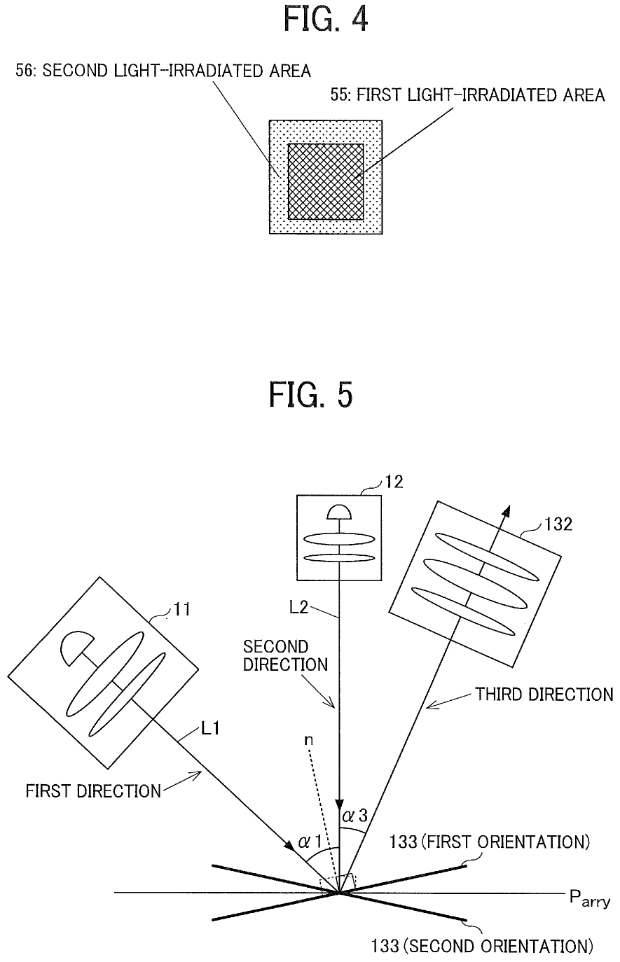

FIG. 4 is an illustration of an irradiation area including a first irradiation area 55 irradiated with the first light L1 (or a first irradiation area 55 formed by the first light L1) and a second irradiation area 56 irradiated with the second light L2 (or a second irradiation area 56 formed by the second light L2). The first light L1 and the second light L2 have different refractive indexes. Although such a mismatch in refractive index between the first light L1 and the second light L2 may cause a slight misalignment between the first irradiation area 55 and the second irradiation area 56, the first light L1 forming the first irradiation area 55 and the second light L2 forming the second irradiation area 56 are substantially coaxial with each other. In the time domain, the time at which the first irradiation area 55 is formed is slightly different from the time at which the second irradiation area 56 is formed, as described above. Although the second irradiation area 56 irradiated with the second light L2 is larger than the first irradiation area 55 irradiated with the first light L1 in FIG. 4, the first irradiation area 55 and the second irradiation area 56 may have the same range because only the irradiation position of the first light L1 is to be recognized.

Referring to FIG. 3, the image-capturing optical system 15A includes an image sensor 16 such as a CMOS on an imaging plane. The image-capturing optical system 15A includes a lens group 151 including one or more lenses; and microlenses 152 disposed upstream of the respective light-receiving elements included in the image sensor 16 in a direction in which light enters the image sensor 16. The image-capturing optical system 15A captures an image of the space including the irradiation area 51 of the object 50. Information detected by the image sensor 16 is fed to the electronic circuit 20. Using the information from the image sensor 16, the control unit 120 implemented by the electronic circuit 20 controls the on and off irradiation states and intensity of each optical modulator and thus enables such adjustment according to the progress of irradiation.

FIG. 5 is an illustration of the relative position between the first illumination optical system 11, the second illumination optical system 12, and the projection optical system 132. A direction in which the first light L1 emitted from the first illumination optical system 11 enters an optical modulator 133 of the optical modulator array 131 is referred to as a first direction. A direction in which the second light L2 emitted from the second illumination optical system 12 enters the optical modulator 133 of the optical modulator array 131 is referred to as a second direction.

A direction in which the first light L1 and the second light L2 are reflected by the optical modulator 133 and directed toward the projection optical system 132 is referred to as a third direction. The first illumination optical system 11, the second illumination optical system 12, and the projection optical system 132 are arranged such that the second direction is between the first direction and the third direction. With this arrangement configuration, the second light L2 as a guide is used together with the first light L1 for irradiation.

The second illumination optical system 12 is disposed such that the second light L2 emitted from the second illumination optical system 12 is incident on an array surface P_{arry} of the optical modulator array 131 at substantially right angle (i.e., the second light L2 emitted from the second illumination optical system 12 substantially perpendicularly enters the array surface P_{arry} of the optical modulator array 131). In other words, the second direction is substantially perpendicular to the array surface P_{arry}. The equation below is satisfied: α1=2×α3 where α1 denotes an angle (an absolute value) between the first direction and the direction perpendicular to the array surface P_{arry} (i.e., the second direction), and α3 denotes an angle (an absolute value) between the third direction and the second direction.

The orientation (angle) of the optical modulator 133 that reflects the first light L1 from the first illumination optical system 11 in the third direction is referred to as a first orientation. In this case, the direction in which the second light L2 enters the optical modulator 133 (i.e., the second direction) is between the third direction and the normal n to the array surface of the optical modulator 133 with the first orientation. Thus, the second light L2 is not reflected by the optical modulator 133 in the third direction. Further, the orientation of the optical modulator 133 that reflects the second light L2 emitted from the second illumination optical system 12 in the third direction is referred to as a second orientation. In this case, the first light L1 is not reflected by the optical modulator 133 in the third direction.

In this example, it is assumed that the first light is UV-B light and the second light L2 is visible light. For example, when the first light L1 is emitted to the irradiation area 51, the second light L2 is reflected by the optical modulator 133 with the second orientation to the irradiation area 51. Immediately after the reflection of the second light L2 to the irradiation area 51, the orientation of the optical modulator 133 is switched from the second orientation to the first orientation, and the first light L1 is reflected by the optical modulator 133 with the first orientation to the irradiation area 51 through the projection optical system 132. With this configuration, the second light L2 in the visible range accurately guides the first light L1 to the irradiation area 51. In some examples, the irradiation position of the first light L1 may be checked, without switching the orientation of an optical modulator 133 of interest, by emitting the first light L1 to the irradiation area and then emitting the second light to an area surrounding the irradiation area irradiated with the first light L1.

The user of the light irradiation device 10A visually observes the visible light serving as a guide to recognize the current position irradiated with the first light (the UV-B light) for irradiation. This configuration enables a higher-accurate irradiation and thus allows the object, or a person to be irradiated, to undergo irradiation with security. By setting the range of wavelengths of the second light L2 to within a range that reduces a biological reaction, a biological reaction on a location that is not irradiated with the first light L1 is reduced.

Further, such irradiation with the second light L2 allows the detection unit 123 of the electronic circuit 20 to detect the number of optical modulators 133 corresponding to the light beams that have failed to hit their intended spots because of the movement of the object. Further, the optical modulator array 131 is controlled by the determination unit 124 and the light control unit 125 to maintain the intended irradiation position of the first light L1. In the optical modulator array 131, each optical modulator 133 may be switched between on and off (or between the first orientation and the second orientation) with a switching frequency per unit time often changeable. This configuration enables a higher accurate irradiation according to the progress of irradiation on the irradiation area.

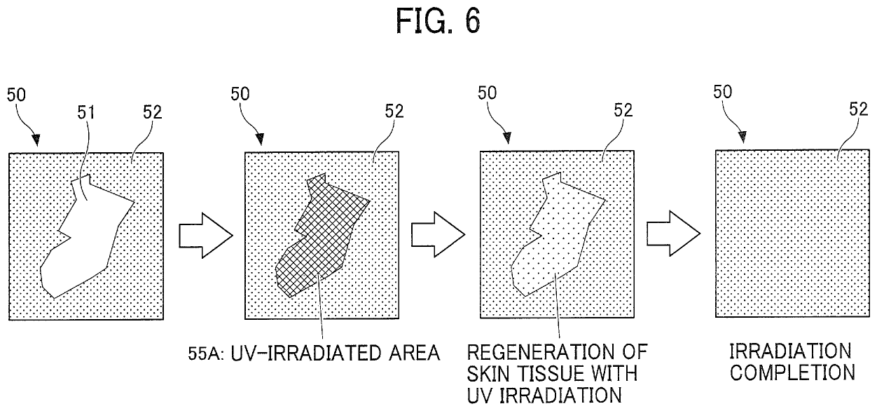

FIG. 6 is an illustration of a healing process using the light irradiation device 10A. For example, when the irradiation area 51 is a vitiligo patch on the skin 52 of the object to be irradiated, each optical modulator 133 is independently controlled to allow the first light L1 (UV-B light) for irradiation to be emitted to the vitiligo patch according to its shape. The first light L1 hitting the white spot causes a chemical reaction between the first light L1 and skin cells to revitalize the skin cells in the irradiated spot. Thus, the irradiation is completed to reduce or eliminate photo-aging.

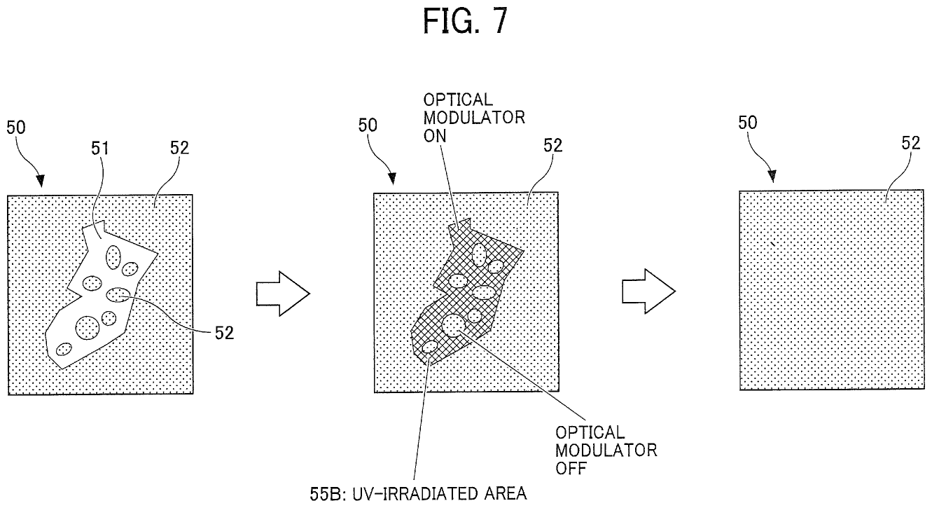

FIG. 7 is an illustration of a healing process using the light irradiation device 10A, according to another embodiment. The light irradiation device 10A according to the first embodiment is also effective for a patchy pattern in which spots (skin spots 52) free of the need for irradiation are included within the irradiation area 51 as the vitiligo patch. The orientations of some optical modulators 133 corresponding to the locations of the skin spots 52 (a second area) free of the need for irradiation within the vitiligo patch are maintained at the second orientation to prevent the first light L1 (the UV-B light) from being emitted to the skin spots 52. The orientations of some other modulators 133 corresponding to the locations of the irradiation area 51 (the intended irradiation spot, a first area to be irradiated) as the vitiligo patch are set to the first orientation so as to emit the first light L1 to the intended irradiation spot. The user of the light irradiation device checks whether the UV-B light is hitting the vitiligo patch (i.e., the intended area to be irradiated, or the irradiation area 51) while visually observing the visible light hitting areas (e.g., the skin spots 52 in and surrounding the irradiation area 51) other than the irradiation area 51. This enables a high-accurate irradiation of the irradiation area 51 while preventing the areas free of the need for irradiation, from being irradiated with the UV-B light.

Further, an exposure dose per unit time is adjusted by changing the durations of the first orientation and the second orientation of the optical modulators 133. As the irradiation proceeds, the state of irradiation may vary between the portions of the skin. In view of such variations, for portions to be irradiated with higher exposure, the durations of the first orientations of some optical modulators corresponding to such portions are increased. For another portion where the vitiligo is healing, the duration of the second orientation may be increased to reduce the exposure dose.

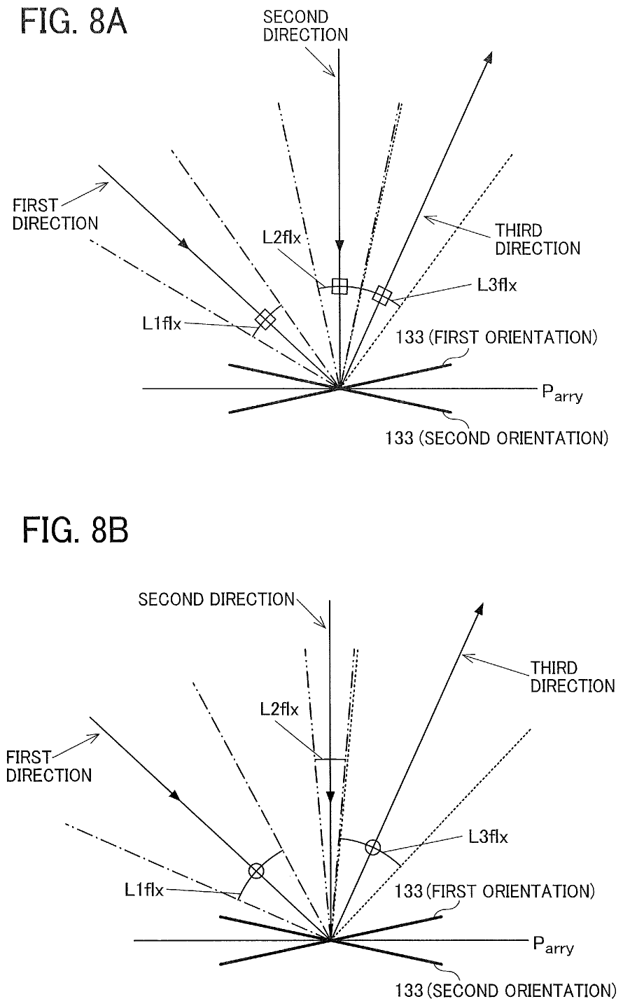

FIG. 8A is an illustration of a configuration that enables maximum amounts of the first light L1 and the second light L2. The first light L1 enters the optical modulators 133 with the first orientation in the first direction and is then reflected to the projection optical system 132 in the third direction. The second light L2 enters the optical modulators 133 with the second orientation in the second direction and is then reflected to the projection optical system 132 in the third direction. Both a first light flux L1flx and a second light flux L2flx have maximum diameters.

FIG. 8B is an illustration of a configuration that enables an increase in the amount of the first light L1. The irradiation efficiency may be desired to be increased by increasing the amount of the first light L1. However, the second light L2, which serves as a guide, does not have to have a high intensity, or brightness. In this case, as illustrated in FIG. 8B, the F-number of each of the first illumination optical system 11 and the projection optical system 132 is reduced (i.e., the numerical aperture NA is increased) to increase the amount of the first light L1. The F-number of the second illumination optical system 12 may be increased (i.e., the numerical aperture NA may be reduced). In other words, the F-number of each of the first illumination optical system 11 and the projection optical system 132 is smaller than the F-number of the second illumination optical system 12. This configuration allows an optimal irradiation to achieve the intended performance. The optical modulator array 131 is tilted relative to the projection optical system 132 (see FIG. 3). The projection optical system 132 may be used to apply the Scheimpflug principle. The Scheimpflug principle is satisfied when the third direction is not perpendicular to the optical modulators 133. In such arrangement in which the optical modulators 133 are not perpendicular to the third direction in which light is projected to the object, the projection optical system 132 disposed perpendicular to the irradiation surface (i.e., the surface to be irradiated) such as skin may cause out-of-focus light at some points within the irradiation area. To avoid such out-of-focus light, the projection optical system 132 is tilted relative to the skin to apply the Scheimpflug principle.

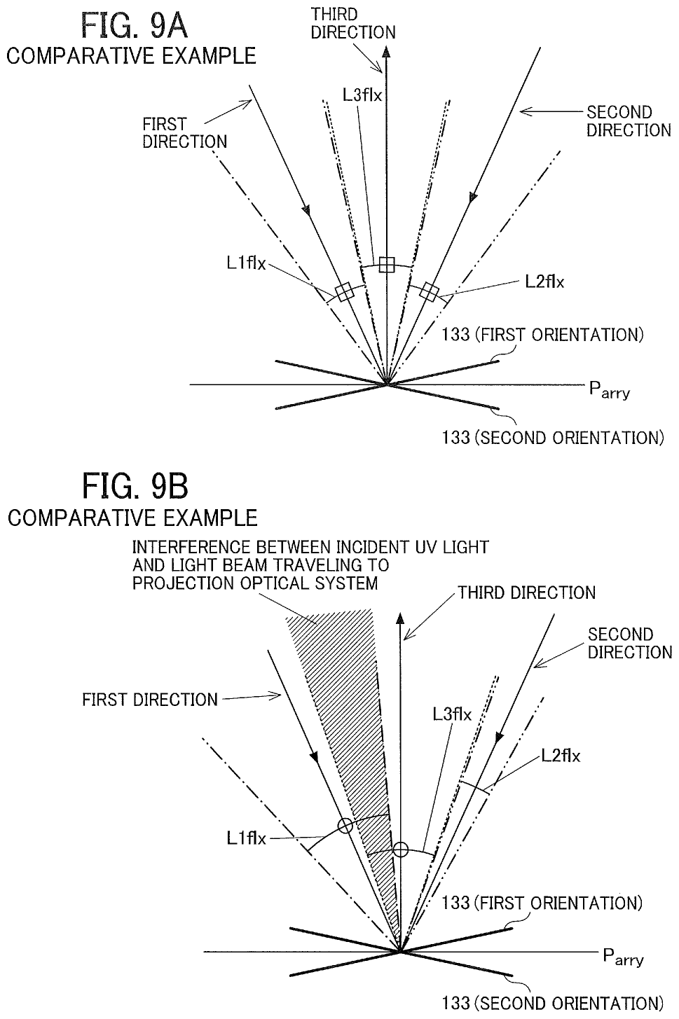

FIG. 9A is an illustration of a configuration that is difficult to increase the amount of the first light, according to a comparative example. In FIG. 9A, a third direction in which light travels to the projection optical system 132 is between the first direction in which light is emitted from the first illumination optical system 11 and the second direction in which light is emitted from the second illumination optical system 12. Both the first light L1 with the maximum amount and the second light L2 with the maximum amount can be reflected to the projection optical system 132.

In FIG. 9B, the F-number of the first illumination optical system 11 is reduced to increase the amount of the first light L1, whereas the F-number of the projection optical system 132 is reduced to capture the amount of the first light L1 reflected by the optical modulators 133. However, as illustrated in FIG. 9B, the light flux L1flx directed in the first direction to enter the optical modulators 133 with the first orientation and the light flux L3flx directed in the third direction after being reflected by the optical modulators 133 interfere with each other. This interference between the light flux L1flx and the light flux L3flx hampers an increase in the amount of light.

In contrast, the configuration according to the FIGS. 8A and 8B increases the amount of the first light L1 as appropriate and achieves irradiation with an optimal exposure dose.

### Second Embodiment

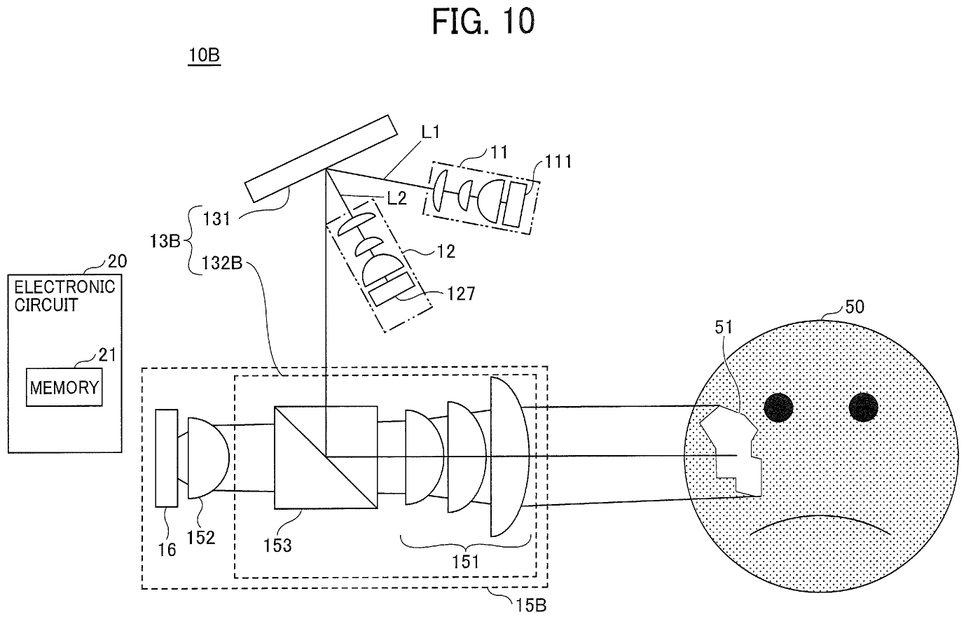

FIG. 10 is an illustration of configurations of the optical systems of a light irradiation device 10B according to a second embodiment. In this example, the light irradiation device 10B is used as a light treatment device. In the second embodiment, a projection optical system 132B is shared by a light emitter 13B and an image-capturing optical system 15B. The projection optical system 132B includes a polarization beam splitter 153 and a lens group 151. The first light L1 output from the first illumination optical system 11 is, for example, s-polarized light. Alternatively, a polarizer may be disposed in a path leading to the projection optical system 132B. The first light L1 is reflected by the optical modulators 133 having the first orientations in the optical modulator array 131 and guided to the polarization beam splitter 153. The s-polarized first light L1 is substantially 100% reflected by the polarization beam splitter 153, passes through the lens group 151, and reaches the irradiation area 51 of the object 50. Thus, the irradiation area 51 is irradiated with the first light L1.

The second light L2 output from the second illumination optical system 12 is reflected by the optical modulators 133 having the second orientations in the optical modulator array 131 and guided to the polarization beam splitter 153. The second light L2 incident on the polarization beam splitter 153 is s-polarized light. The s-polarized light may be emitted from the second illumination optical system 12, or a polarizer may be disposed in a path leading to the polarization beam splitter 153. The s-polarized second light L2 is substantially 100% reflected by the polarization beam splitter 153, and projected into the same space as the first light L1 through the lens group 151.

The image-capturing optical system 15B is coaxial with the projection optical system 132B. The image-capturing optical system 15B captures an image of the space including the irradiation area 51. Light diffused and reflected by the object 50 is unpolarized light. The diffused and reflected light is converged by the lens group 151. The light component transmitted through the polarization beam splitter 153 is condensed by a microlens 152 and detected by an image sensor 16.

The configuration according to the second embodiment allows lower manufacturing cost of the light irradiation device 10B and achieves miniaturization. When it is desired to give priority to the flexibility of design and the improvement of the performance, the image-capturing optical system 15 and the projection optical system 132 may be separate from each other as in the light irradiation device 10 according to the first embodiment.

### Third Embodiment

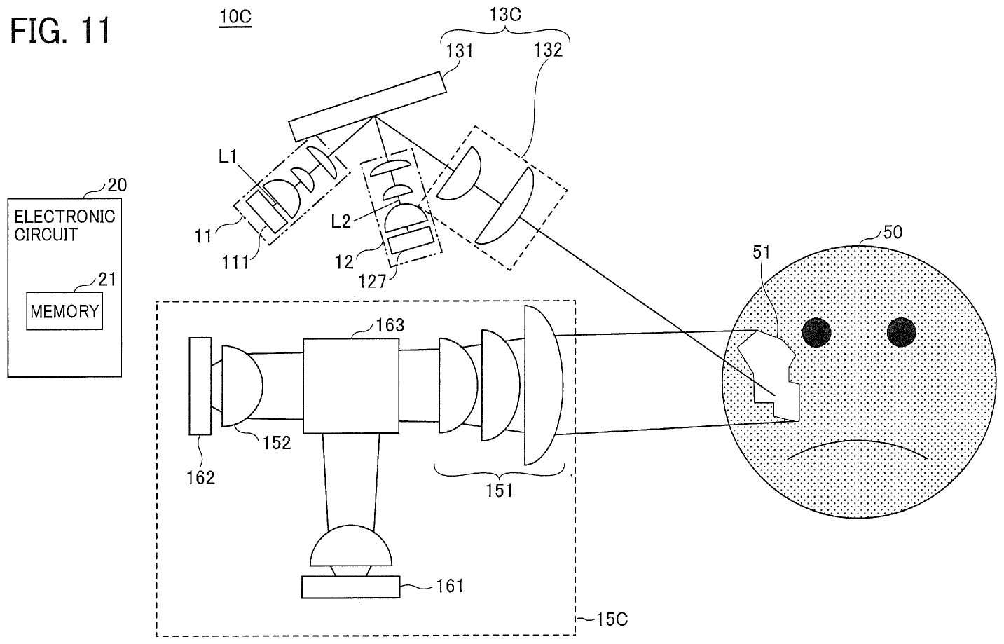

FIG. 11 is an illustration of configurations of the optical systems of a light irradiation device 10C according to a third embodiment. The light irradiation device 10C is used as a light treatment device. In the third embodiment, a first image sensor 161 having sensitivity to the first light and a second image sensor 162 having sensitivity to the second light L2 are used. The first image sensor 161 has sensitivity to, for example, UV-B light, and the second image sensor 162 has sensitivity to visible light.

The image-capturing optical system 15C includes a dichroic beam splitter 163 that separates incident light to the first image sensor 161 and the second image sensor 162. The dichroic beam splitter 163 may be a prism beam splitter or a plate beam splitter as long as the dichroic beam splitter 163 is capable of separating the incident light into the UV-B light and the visible light in this example.

The image processing unit 122 of the control unit 120, which is implemented by the electronic circuit 20, generates an image that allows visual observation of the first light L1, based on the electrical signal output from the first image sensor 161. In this case, the light irradiation device 10C is provided with a display or connected to an external display device, to which image data is output, so as to display the UV-B light with a recognizable color.

In other words, the image formed with the UV-B light and the visible light is displayed. This allows recognition as to which locations are irradiated with the UV-B light from the screen. When an image sensor having sensitivity to both the first light and the second light is used, one image sensor 16 is used. In this case, the quantum efficiency QE₂₈₀ of the image sensor 16 at a wavelength of 280 nm is preferably 20% or greater to allow output sufficient to achieve intended performance.

Although the present disclosure has been described above based on specific configuration examples, the present disclosure is not limited to these configuration examples. Alternatively, the first light may be infrared light instead of ultraviolet light. Since infrared light is also invisible to human eyes, light in a visible range may be used as a guide (or guide light) together with infrared light for irradiation. This configuration also allows emission of the guide light while switching between ON and OFF (i.e., the first orientation and the second orientation) of each optical modulator 133 at high speed and thus achieves a higher-accurate irradiation of the intended irradiation area with infrared light irrespective of the shape of the irradiation area 51. Irrespective of which type of light is used as the first light, ultraviolet light or infrared light, the light irradiation device is used as a light treatment device.

Using visible light together with near-infrared light allows detection of misalignment of the irradiation position of the near-infrared light due to the movement of the object 50 by monitoring the visible light. Based on the detection result, the optical modulators 133 can be quickly controlled to correct the irradiation position of the infrared light. When the light irradiation device is used as a light treatment device, the amount of near-infrared light is adjusted according to the degree of healing of the affected part.

A light irradiation method according to an embodiment includes capturing an image of a full field including an irradiation area on an object irradiated with first light and second light coaxially emitted to the object, the first light and the second light having wavelengths different from each other; detecting a state of the irradiation area based on the captured image; according to the state of the irradiation area detected in the detecting, determining an orientation of each optical modulator of an optical modulator array that emits the first light and the second light to the object; controlling each optical modulator of the optical modulator array to have the orientation determined in the determining; controlling a first light source to emit the first light in a first direction and a second light source to emit the second light in a second direction to the optical modulator array with each optical modulator controlled to have the orientation determined in the determining; and emitting the first light reflected in a third direction by each optical modulator and the second light reflected in the third direction by each optical modulator to irradiate the object, the third direction being different from each of the first direction and the second direction.

Further, in the light irradiation method according to an embodiment, the controlling of each optical modulator of the optical modulator array to have the orientation determined in the determining involves controlling each optical modulator to have a first orientation before controlling the first light source to emit the first light in the first direction to each optical modulator. Further, the controlling of each optical modulator of the optical modulator array to have the orientation determined in the determining involves controlling each optical modulator to have a second orientation before controlling the second light source to emit the second light in the second direction to each optical modulator.

The light irradiation method according to an embodiment further includes controlling some optical modulators corresponding to a first area on the object to be irradiated to have the first orientation and controlling some other optical modulators corresponding to a second area other than the first area on the object to have the second orientation among the multiple optical modulators so as to emit the first light to the first area with the second light as a guide for the first light. The object is skin including the first area and the second area.

The light irradiation method according to an embodiment further includes repeating operations of the capturing, the detecting, the determining, the controlling of each optical modulator, the controlling of the first light source and the second light source, and the emitting.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention.

Any one of the above-described operations may be performed in various other ways, for example, in an order different from the one described above.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), a digital signal processor (DSP), a field programmable gate array (FPGA), and conventional circuit components arranged to perform the recited functions.

1. A light irradiation device comprising:
a first illumination optical system to emit first light that causes a chemical reaction on an object;
a second illumination optical system to emit second light having a wavelength different from a wavelength of the first light;
a light emitter to emit the first light from the first illumination optical system and the second light from the second illumination optical system to the object in space shared by the first light and the second light, to irradiate an irradiation area on the object;
an image-capturing optical system to capture an image of the space including the object; and
circuitry configured to:
detect a state of the irradiation area based on the image captured by the image-capturing optical system; and
control at least one of the first light and the second light based on the detected state.

2. The light irradiation device according to claim 1,
wherein the light emitter includes:
an optical modulator array including an array of multiple optical modulators to reflect each of the first light and the second light incident thereon in a reflection direction, each optical modulator having an orientation that is changeable to switch the reflection direction between a predetermined direction and another direction so as to reflect the first light and the second light in the predetermined direction; and
a projection optical system to emit to the object, the first light reflected by each optical modulator in the predetermined direction and the second light reflected by each optical modulator in the predetermined direction.

3. The light irradiation device according to claim 2,
wherein the first illumination optical system emits the first light in a first direction to the optical modulators,
wherein the second illumination optical system emits the second light in a second direction to the optical modulators,
wherein the predetermined direction includes a third direction, and
wherein the second direction is between the first direction and the third direction.

4. The light irradiation device according to claim 3,
wherein the orientation of each optical modulator includes:
a first orientation to reflect the first light in the third direction; and
a second orientation to reflect the second light in the third direction, and
wherein the circuitry controls the orientation of each optical modulator based on the detected state.

5. The light irradiation device according to claim 4,
wherein the circuitry controls a duration of each of the first orientation and the second orientation based on the detected state.

6. The light irradiation device according to claim 3,
wherein the second direction is perpendicular to an array surface of the optical modulator array, and
wherein an equation below is satisfied:
α1=2×α3
where
α1 denotes an absolute value of an angle between the second direction and the first direction, and
α3 denotes an absolute value of an angle between the second direction and the third direction.

7. The light irradiation device according to claim 3,
wherein an F-number of each of the first illumination optical system and the projection optical system is smaller than an F-number of the second illumination optical system.

8. The light irradiation device according to claim 1,
wherein the first light is ultraviolet light or infrared light.

9. The light irradiation device according to claim 1,
wherein the second light is visible light.

10. The light irradiation device according to claim 1,
wherein the light irradiation device includes a light treatment device.

11. A light irradiation method comprising:
capturing an image of a full field including an irradiation area on an object irradiated with first light and second light coaxially emitted to the object, the first light and the second light having wavelengths different from each other;
detecting a state of the irradiation area based on the captured image;
determining an orientation of each optical modulator of an optical modulator array that emits the first light and the second light to the object according to the state of the irradiation area detected in the detecting;
controlling each optical modulator of the optical modulator array to have the orientation determined in the determining;
controlling a first light source to emit the first light in a first direction and a second light source to emit the second light in a second direction to the optical modulator array with each optical modulator controlled to have the orientation determined in the determining; and
emitting the first light reflected in a third direction by each optical modulator and the second light reflected in the third direction by each optical modulator to irradiate the object, the third direction being different from each of the first direction and the second direction.

12. The light irradiation method according to claim 11,
wherein the controlling of each optical modulator of the optical modulator array to have the orientation determined in the determining involves:
controlling each optical modulator to have a first orientation before controlling the first light source to emit the first light in the first direction to each optical modulator; and
controlling each optical modulator to have a second orientation before controlling the second light source to emit the second light in the second direction to each optical modulator.

13. The light irradiation method according to claim 12, further comprising:
controlling some optical modulators corresponding to a first area on the object to be irradiated to have the first orientation and controlling some other optical modulators corresponding to a second area other than the first area to have the second orientation so as to emit the first light to the first area with the second light as a guide for the first light,
wherein the object is skin including the first area and the second area.

14. The light irradiation method according to claim 11, further comprising repeating the capturing, the detecting, the determining, the controlling of each optical modulator, the controlling of the first light source and the second light source, and the emitting.
